(19) United States
(12) Plant Patent Application Publication
(10) Pub. No.: US 2020/0196499 P1
(43) Pub. Date: Jun. 18, 2020

(54) Kiwi plant named 'AC 501 022'

(50) ;

(71) Applicant: Università degli Studi di Udine, Udine (IT)

(72) Inventors: Raffaele Testolin, Udine (IT); Guido Cipriani, Faedis (IT)

(73) Assignee: Università degli Studi di Udine, Udine (IT)

(21) Appl. No.: 16/602,861

(22) Filed: Dec. 11, 2019

(30) Foreign Application Priority Data
Dec. 14, 2018 (QZ) 2018/3379

(51) Int. Cl. A01H 6/00

(52) U.S. Cl. PLT/156

(57) ABSTRACT

A new female kiwi plant named ‘AC 501 022’, distinguished by its oblong fruit with dark brown hairless skin and yellow flesh. The fruit is very sweet with low acidity, and matures relatively early.

Genus and species: Actinidia chinensis Planch.

Variety denomination: ‘AC 501 022’.

### BACKGROUND AND SUMMARY OF THE NEW VARIETY

The invention relates to a new variety of kiwi plant (Actinidia chinensis Planch.) named ‘AC 501 022,’ which originated in the course of a plant breeding program at the University of Udine, Italy. Female parent ‘Ac328.26 (not patented) was crossed with male parent ‘A134.41’ (not patented), and ‘AC 501 022’ was selected from the resulting seedlings. The new variety has been asexually reproduced by grafting and by in vitro propagation, and has been found to remain true-to-type over successive generations.

‘AC 501 022’ is distinguished from similar variety ‘Soreli’ (U.S. Plant Pat. No. 21,552) by the characteristics set forth in Table 1 below.

TABLE 1
Comparison of ‘AC 501 022’ to ‘Soreli’

| | ‘AC 501 022’ | ‘Soreli’ |
|---|---|---|
| Plant vigor | Strong | Weak |
| Density of hair on | Very sparse | Medium |
| young shoot | | |
| Color of stem shoot | Grey brown | Purple brown |
| on sunny side | | |
| Shape of leaf apex | Retuse | Acuminate |
| Leaf basal lobes | None | Slightly |
| | | overlapping |

### DESCRIPTION OF THE PHOTOGRAPHS

FIG. 1 shows a branch, fruits and leaves of ‘AC 501 022’;

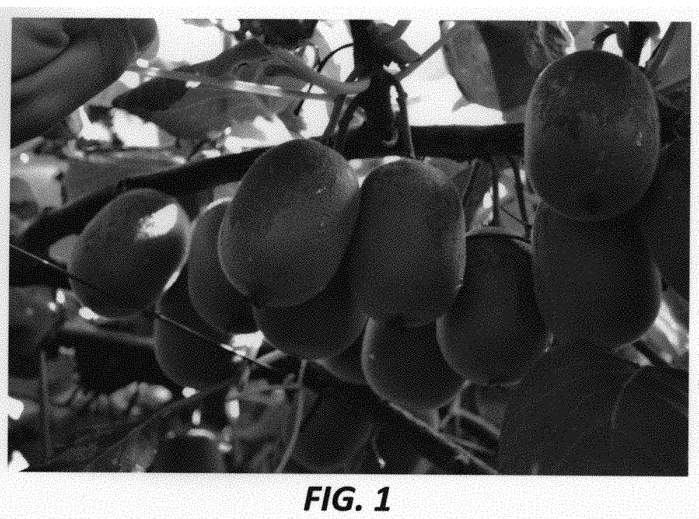

FIG. 2 shows a shoot and young leaves of ‘AC 501 022’;

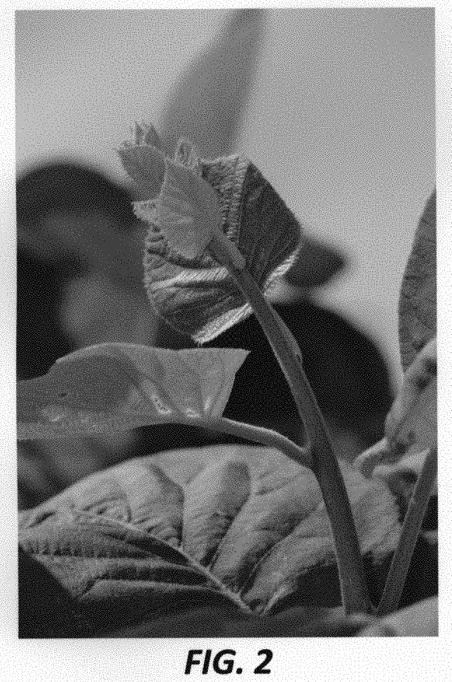

FIG. 3 shows a mature leaf of ‘AC 501 022’; and,

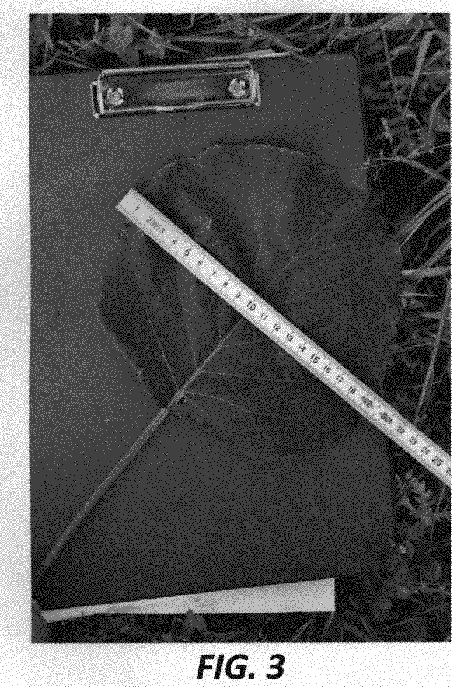

FIG. 4 shows whole and sectioned fruit of ‘AC 501 022’.

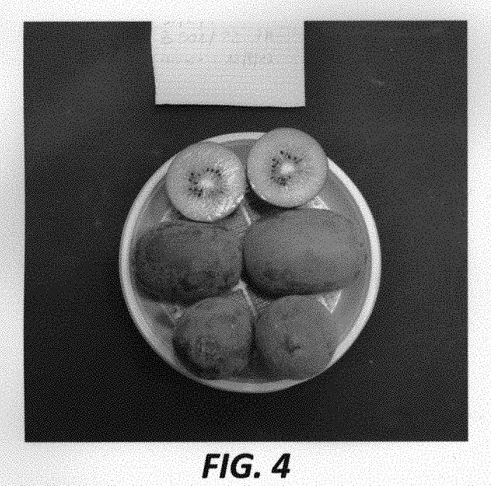

### DETAILED BOTANICAL DESCRIPTION

The characteristics described herein will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Plant sex: Female.
Self-fruit: setting Absent.
Plant vigor: Strong.
Density of hairs on young shoot: Very sparse.
Anthocyanin: coloration of growing tip on young shoot Weak.
Stem:
Thickness: Medium.
Color of shoot on sunny side: Grey brown.
Texture of bark: Smooth.
Density of hairs: Absent or sparse.
Size of lenticels: Small.
Number of lenticels: Medium.
Prominence of bud support: Very weak.
Presence of bud cover: Absent.
Size of hole in bud cover: Medium.
Leaf scar: Moderately depressed.
Pith: Lamellate.
Leaf:
Shape: Obovate.
Length/width ratio: Intermediate.
Shape of apex: Retuse.
Basal lobes: None.
Number of ciliate serrations: Medium.
Density of hairs on upper side: Medium.
Density of hairs on lower side: Sparse.
Intensity of green color of upper side: Medium.
Color of lower side: Light green.
Variegation: Absent.
Length of petiole relative to blade: Large.
Anthocyanin coloration of upper side: Medium.
Flowers:
Inflorescence type: Dichasium.
Number of flowers: Few.
Flower bud: position of first spike: Medium.
Number of sepals: Medium.
Main color of sepals: Brown.
Density of sepal hairs: Absent or sparse.
Diameter: Medium.
Arrangement of petals: Overlapping.
Shape in profile: Convex.
Number of styles: Medium.
Attitude of styles: Semi-erect.
Petal: color on adaxial side: Yellow.
Petal: shading of main color: Lighter towards apex.
Anther color: Yellow orange.
Pollenizers: Belen, Zuva, Matua (not patented).
Fruit:
Weight: Medium.
Width: Medium.
Length/width ratio: Medium.
Shape: Oblong.
Shape in cross section at median: Circular.
Stylar end: Strongly depressed.
Presence of calyx ring: Medium.
Shape of shoulder at stalk end: Weakly sloping.
Length of stalk: Medium.
Length of stalk relative to length of fruit: Medium.
Conspicuousness of lenticels on skin: Weak.
Hairiness of skin: Absent.
Color of skin: Dark brown.
Adherence of skin to flesh: Strong.
Color of outer pericarp: Medium yellow.
Color of locules: Medium yellow.
Width of core relative to fruit: Small.
General shape of core in cross section: Circular.
Color of core: Yellow white.
Sweetness: High.
Acidity: Low.
Timing:
Time of vegetative bud burst: Early.
Time of beginning of flowering: Medium.
Time of maturity for harvest: Early.

We claim:

1. A new and distinct kiwi plant substantially as described and illustrated herein.
